(19) United States
(12) Patent Application Publication
(10) Pub. No.: US 2024/0309169 A1
(43) Pub. Date: Sep. 19, 2024

(54) Method for Producing Monomers And/or Oligomers From a Polymer

(71) Applicant: COVESTRO DEUTSCHLAND AG, Leverkusen (DE)

(72) Inventors: Matthias Leven, Köln (DE); Norah Heinz, Leverkusen (DE); Jens Langanke, Mechernich (DE); Torsten Heinemann, Leichlingen (DE); Tzvetan Mihaylov, Leuven (BE); Kai Laemmerhold, Odenthal (DE); Nicklas Deibl, Aachen (DE); Tugrul Nalbantoglu, Aachen (DE)

(21) Appl. No.: 18/576,397

(22) PCT Filed: Jul. 4, 2022
(86) PCT No.: PCT/EP2022/068428

§ 371 Date: Jan. 4, 2024

(30) Foreign Application Priority Data
Jul. 5, 2021 (EP) 21183712.5

(51) Int. Cl. C08J 11/22; C07C 209/62; C08J 11/14; C08J 11/16

(52) U.S. Cl. CPC C08J 11/22; C07C 209/62; C08J 11/14; C08J 11/16; C08J 2300/106

(57) ABSTRACT

The invention relates to a method for producing monomers and/or oligomers from a polymer that includes a structural unit having a nitrogen-carbonyl carbon bond. The nitrogen-carbonyl carbon bond is broken in a chemical reaction with formaldehyde or paraformaldehyde as the activating reagent. The reaction takes place using a Lewis acid acting as a catalyst. The invention also relates to monomers, in particular a carboxylic acid monomer and an aldimine monomer or an amine monomer, obtained or produced from this method.

### CROSS-REFERENCE TO RELATED APPLICATIONS

This application is the United States national phase of International Application No. PCT/EP2022/068428 filed Jul. 4, 2022, and claims priority to European Patent Application No. 21183712.5 filed Jul. 5, 2021, the disclosures of which are hereby incorporated by reference in their entireties.

### BACKGROUND

### Technical Field

The present invention relates to a method for producing monomers and/or oligomers from a polymer comprising a structural unit having a nitrogen-carbonyl carbon bond.

### Description of Related Art

The invention also relates to monomers, in particular a carboxylic acid monomer and an aldimine monomer or an amine monomer, obtainable or produced by this method.

There are a large number of synthetic polymers (including plastics) that are used in a wide variety of areas, for example in the packaging, automotive and electronics industries, but also in construction and in medicine. Plastics are characterized on the one hand by their diverse mechanical, physical and chemical properties and on the other hand by their relatively low production costs. As an alternative to new syntheses, there is growing interest in recycling technologies which, on the one hand, enable recycling of the plastics and, on the other hand, are economically competitive with the new syntheses.

One of the most industrially important synthetic polymers is polyamide. Polyamides are polymers having regularly repeating amide bonds along a polymer backbone. Such amide bonds can be formed by condensation of a carboxylic acid and an amine to form a nitrogen-carbonyl carbon bond. Depending on the number of monomers linked together and forming the polymer, a polymer can have a large number of structural units having such nitrogen-carbonyl carbon bonds. Aminocarboxylic acids, lactams, diamines and/or dicarboxylic acids, inter alia, are used as monomers for producing polyamides. The two most commonly used polyamides are polyamide 6.6 and polyamide 6 (also known as nylon 6.6 and nylon 6, respectively). Polyamide 6.6 is produced from hexamethylenediamine and adipic acid. It is formed by polycondensation with elimination of water. Polyamide 6, in turn, is formed by ring-opening polymerization of ε-caprolactam with water as starter. Due to their strength and toughness, polyamides are often used as construction materials.

Another synthetic polymer also industrially important is polyurethane. Polyurethanes are characterized by regularly repeating urethane groups. As with amide bonds, part of these urethane groups is a nitrogen-carbonyl carbon bond, while urethane groups, in contrast to amide groups, have an additional oxygen atom. Polyurethanes are formed in the polyaddition reaction of polyols with polyisocyanates. The reaction of dialcohols (also called diols) with diisocyanates results in linear polyurethanes, whereas the reaction of, for example, triisocyanate-diisocyanate mixtures with triol-diol mixtures results in crosslinked polyurethanes. Depending on the degree of crosslinking and the isocyanate and alcohol components used, polymers having different properties result. Thus, polyurethanes can form thermosets, thermoplastics or elastomers. Polyurethanes are most commonly used to produce soft or rigid foams. However, they can also be used as compressible molding compositions, as casting resins (isocyanate resins), as (textile) elastic fibers, as paints and as adhesives.

As already mentioned at the outset, there is great interest in recycling synthetic polymers and in particular in treating them by means of chemical recycling processes in such a way that the monomers used in the synthesis or at least oligomers and low-molecular weight chemical compounds can be recovered. Ideally, new polymers can then be synthesized from these monomers/oligomers again.

Such chemical recycling processes are used for both polyamides and polyurethanes. Depending on the polymer to be recycled, different processes are used to break down the polymer structures.

For example, EP 1 801 101 A1 discloses a process for producing monomers from a polymer comprising a structural unit having a nitrogen-carbonyl carbon bond. A process for producing monomers from polyamides is disclosed. In the process, the nitrogen-carbonyl carbon bond is cleaved in a chemical reaction with an alcohol having two or more carbon atoms as the activating reagent. The reaction does not require a catalyst and is carried out at a reaction temperature of ≥350° C. The process is particularly suitable for polyamide 6, from which up to 97% of the ε-caprolactam monomer are recovered by means of the process, depending on the alcohol used.

A process for producing monomers from polyamide 6 is also known from U.S. Pat. No. 5,668,277 A. In the process, polyamide 6 is cleaved using ammonia, an amine or a mixture thereof as activating reagent. The reaction is carried out at a reaction temperature between 200 and 400° C. and at a pressure of about 0.5 to 5 atm. Reaction products include ε-caprolactam and ε-caprolactam precursor molecules such as ε-aminocaproic acid, which can be used directly for the new synthesis of polyamides.

For polyamide 6.6, a complex process is known from U.S. Pat. No. 4,605,762 A, in which the monomers are recovered from the polyamide 6.6 by hydrolysis at a reaction temperature of 200 to 300° C. and a pressure of at least 15 atm.

The scientific publication “A new approach to chemical recycling of polyamide 6.6 and synthesis of polyurethanes with recovered intermediates” by Datta et al., J Polym Environ 26, 4415-4429 (2018) discloses another process for the depolymerization of polyamide 6.6. In this case, polyamide 6.6 is reacted with ethylene glycol or a mixture as ethylene glycol and triethylenetetramine as activating reagent in the presence of a catalyst. A mixture of low molecular weight compounds is formed which includes, inter alia, hexamethylenediamine, a substance having terminal β-hydroxyethyl ester groups, esters and amines. The reaction products were successfully able to be used for the new synthesis of polyurethanes.

The scientific publication “Hydrogenative Depolymerization of Nylons”, J. Am. Chem. Soc. 2020, 142, 33, 14267-14275 describes a process for depolymerization of polyamides by hydrogenation with gaseous hydrogen.

U.S. Pat. No. 2,430,860 A describes a process for modifying polyamides using formaldehyde as crosslinker, together with crosslinkable compounds. Here, in the presence of strong Brönsted acids having a defined dissociation constant, unwanted chain breaks occur, which result in impairment of the polymer quality. A depolymerization to give oligomers or monomeric polyamide units, or a process for obtaining these, is not disclosed. Patent specification DE 2430 222 C describes a similar process but in which there is no mention of any damage to the polyamides.

Even if processes for the depolymerization of synthetic polymers are already known that involve the recovery of monomers and/or oligomers, there is still a great need for alternative, less complex, environmentally friendly and cost-effective processes for producing monomers and/or oligomers from polymers.

### SUMMARY

It is therefore the object of the present invention to specify a method for producing monomers and/or oligomers from a polymer comprising a structural unit having a nitrogen-carbonyl carbon bond, which allows less complicated, more environmentally friendly and more cost-effective polymer recycling.

This object is achieved by a method for producing monomers and/or oligomers from a polymer comprising a structural unit having a nitrogen-carbonyl carbon bond, wherein the nitrogen-carbonyl carbon bond is cleaved in a chemical reaction with formaldehyde or paraformaldehyde as activating reagent, and wherein the reaction is carried out using a Lewis acid acting as catalyst.

Furthermore, the present invention is based on the object of producing monomers, in particular a carboxylic acid monomer and an aldimine monomer or an amine monomer, by means of a recycling process from polymers, with less complexity compared to the prior art, in a more environmentally friendly manner and at lower cost.

This object is achieved by monomers, in particular a carboxylic acid monomer and an aldimine monomer or an amine monomer, which are obtainable or have been produced from such a process.

It should be noted that the features listed individually in the claims can be combined with one another in any technically useful way (even across category boundaries, for example between method and device) and demonstrate further configurations of the invention. The description additionally characterizes and specifies the invention.

It should also be noted that a conjunction “and/or” used herein, between two features and linking them to one another is always to be interpreted in such a way that in a first configuration of the subject matter of the invention only the first feature can be present, in a second configuration only the second feature can be present and in a third configuration both the first and the second feature can be present.

Preferred configurations of the invention are the subject matter of the dependent claims.

### DESCRIPTION

It is provided in accordance with the invention that monomers and/or oligomers are produced from a polymer, which has a structural unit having a nitrogen-carbonyl carbon bond, by reacting the polymer in a chemical reaction with formaldehyde or paraformaldehyde as activating reagent. A Lewis acid serves as catalyst here. The chemical reaction that takes place during the process results in the bond between the nitrogen atom and the carbonyl carbon atom of the nitrogen-carbonyl carbon bond being cleaved, so that subsequently monomers and/or oligomers can be recovered from the polymers used for the process. The term “structural unit” refers to functional groups which, in particular, link the repeating units present in the polymer to one another.

In the context of the present invention, a “Lewis acid” is to be understood to mean an electrophilic electron pair acceptor capable of attracting electron pairs. The Lewis acids include, inter alia, protons and metal cations.

Synthetic polymers are particularly suitable as a reaction starting material (i.e. as a starting substance) for the method according to the invention. Particularly suitable for use may be aliphatic polyamides or aliphatic polyurethanes. In principle, it is conceivable to use the method according to the invention with unmixed polymer waste, which can provide the reaction starting materials. However, it also seems possible to use the method according to the invention with waste mixtures of different polymers, although with the need for subsequent purification and with a higher probability of undesired side reactions. As a rule, a polymer waste mixture will therefore be separated or purified before use as a reaction starting material in the present method, so that as far as possible single variety polymers are present as reaction starting materials. Polymer waste can be separated mechanically, physically or chemically, i.e. using appropriate sorting and purification processes.

A significant advantage of the method according to the invention is that it is a less complex, environmentally friendly, cost-effective and efficient alternative to the methods already known from the prior art for producing monomers and/or oligomers from a polymer comprising a structural unit having a nitrogen-carbonyl carbon bond.

In the process, formaldehyde or paraformaldehyde may be used stoichiometrically. The term “stoichiometric” is to be understood here as meaning that the amount of formaldehyde used or of the repeating units of paraformaldehyde used is identical to the amount of repeating units of the polymer used or is a multiple or a whole multiple of the latter. The stoichiometric use of formaldehyde or paraformaldehyde ensures that, for all nitrogen-carbonyl carbon bonds in the polymer, at least one formaldehyde molecule or formaldehyde radical is available which can cleave the bond between the nitrogen atom and the carbonyl carbon atom of the nitrogen-carbonyl carbon bond. It is therefore to be expected that when formaldehyde or paraformaldehyde is used stoichiometrically, the yield of monomers and/or oligomers cleaved from the polymer is maximized.

Alternatively, formaldehyde or paraformaldehyde may also be used substoichiometrically. “Substoichiometric” in turn means that the amount of formaldehyde used or of the repeating units of paraformaldehyde used is only a fraction of the amount of repeating units of the polymer used. This has the advantage that in this way a smaller amount of activating reagent is required when carrying out the method and costs can therefore be saved. Furthermore, a lower use of materials is associated with an improved environmental balance.

Preference can be given to using a metal catalyst as Lewis acid. The metal catalyst may particularly preferably comprise a metal from the following group: aluminum, boron, bismuth, cerium, iron, copper, lanthanum, magnesium, tin, titanium, zirconium or zinc. The metals can preferably be used in the form of their chloride salts. The use of other salts is also conceivable. Preferred oxidation states of the aforementioned metal catalysts are aluminum(III), boron(III), bismuth(III), cerium(III), iron(III), copper(II), lanthanum(III), magnesium(II), tin(IV), titanium(IV), zirconium(IV) or zinc(II).

According to a further configuration of the invention, a metal triflate compound, preferably bismuth triflate, may be used as metal catalyst. Triflate compounds have been used as Lewis acids in many reactions with increasing success in recent years. The advantage of these salt compounds is the extreme stability of the triflate anion. Even when dissolved in water, the triflate anion does not react further, in contrast to many other classic Lewis acids. The result of this is that the chemical reaction taking place when carrying out the method can be better controlled and undesired side reactions can be better avoided.

In addition to bismuth triflate, suitable metal triflate compounds are, for example, zinc triflate, aluminum triflate, cerium(III) triflate, scandium triflate, neodymium triflate, samarium triflate, yttrium triflate, lanthanum triflate or gadolinium triflate.

In addition to bismuth triflate, particularly preferred metal triflate compounds are thus zinc triflate, aluminum triflate and cerium(III) triflate.

Furthermore, the chemical reaction can be carried out in an organic solvent, in particular in a polar organic solvent. If a solvent is required, for example because the polymer used, the activating reagent and/or the catalyst are in the solid phase, an organic solvent serves to bring the molecules involved in the reaction into better contact with one another by dissolving one or more of the compounds used completely or at least partially. The use of a polar organic solvent has the advantage that metal triflate compounds, such as can be used in the invention as catalytically active Lewis acid, readily dissolve therein. Which (polar) organic solvents can be used for the method depends heavily on the polymer used, the activating reagent and the catalyst selected. Examples of suitable polar organic solvents are dioxolane, tetrahydrofuran, dioxane, dimethoxyethane, o-dichlorobenzene, dimethyl sulfoxide, dimethylformamide, methanol, ethanol, ethylene glycol, acetic acid, trioxane or chloroform, preferably trioxane or chloroform. Alternatively or additionally, in addition to the polar organic solvents, water can also be used as solvent for the method according to the invention.

In addition, water can be used as additive for the method according to the invention. The use of water as additive results in the reaction equilibrium of the chemical reaction taking place when carrying out the method to be shifted in favor of the reaction products. This is because water promotes the cleavage of nitrogen-carbonyl carbon bonds and thus the formation of further monomers and/or oligomers. If a metal triflate compound is used as catalyst in addition to the additive water, the triflate anion formed when water is added remains stable in the solution and advantageously does not react further. The water can also be added continuously to enhance hydrolysis without unduly quenching the Lewis acid. In other words, the Lewis acid used in the context of the present invention is not neutralized by the presence of water and/or basic amines.

According to a further configuration of the invention, the monomers and/or oligomers can be produced in a reaction comprising a first and a second reaction stage. The first reaction stage can be carried out here at a first reaction temperature of ≤100° C., preferably ≤80° C., particularly preferably ≤60° C. The second reaction stage, in turn, can be carried out at a second reaction temperature of ≤200° C., preferably ≤180° C., particularly preferably ≤165° C. Alternatively, the first and the second reaction stage can both be carried out at a uniform reaction temperature, ideally at the second reaction temperature, i.e. at ≤200° C., preferably at ≤180° C., particularly preferably at ≤165° C. It has been found that the first reaction stage already takes place at a lower reaction temperature. However, the first and second reaction stages can be carried out at the same reaction temperature, which simplifies carrying out the method, since the reaction temperature only has to be adjusted once. Compared to other methods known from the prior art for producing monomers and/or oligomers from polymers, the reaction temperatures used here are comparatively low (mild reaction conditions). Accordingly, the method described here is more energy-efficient and more environmentally friendly than other (chemical) recycling processes for polymers.

A hemiaminal can be formed in the first reaction stage. This is particularly the case when a polyamide or a polyurethane is used as polymer.

A carboxylic acid and an aldimine can in turn be formed in the second reaction stage. This is the case when a polyamide is used as polymer. A carboxylic acid and an aldimine are particularly simple structures that can easily be used for new synthetic reactions.

The aldimine can then be further converted to an amine. Compared to the aldimine, an amine is an even simpler structure that is also best suited for recycling in new synthetic reactions.

The process can produce aliphatic, araliphatic or cycloaliphatic amines. The amine produced can include, in particular, an amine from the following group: tetramethylenediamine, pentamethylenediamine (PDA), hexamethylenediamine (HDA), 2-methyl-1,5-diaminopentane, 1,5-diamino-2,2-dimethylpentane, 2,2,4- or 2,4,4-trimethyl-1,6-diaminohexane, 1,10-diaminodecane, 1,3- and 1,4-diaminocyclohexane, 1,3- and 1,4-bis(aminomethyl)cyclohexane (H6-XDA), 1-isocyanato-3,3,5-trimethyl-5-isocyanatomethylcyclohexane (isophoronediamine; IPDA), 2,4′- and 4,4′-diaminodicyclohexylmethane, 1,3- and 1,4-bis(aminomethyl)benzene (XDA) or bis (aminomethyl)norbornane (NBDA). The amine produced particularly preferably comprises an amine from the following group: pentamethylenediamine (PDA), hexamethylenediamine (HDA), isophoronediamine (IPDA), 2,4′- and 4,4′-diaminodicyclohexylmethane, 1,3- and 1,4-diaminocyclohexane, 1,3- and 1,4-bis (aminomethyl)cyclohexane (H6-XDA), 1,3- and 1,4-bis(aminomethyl)benzene (XDA) or bis(aminomethyl)norbornane (NBDA). A condition for the preparation of the amines mentioned is that the polymers used have appropriate repeating units which allow the amines mentioned to be synthesized.

Alternatively or additionally, the method can be used to produce alcohols and isocyanates. These can be both monomers and oligomers. Alcohol and isocyanate can be formed when a polyurethane polymer is used as polymer. Alcohols and isocyanates are also simple structures that are well suited to new syntheses. An example of a possible reaction product when a corresponding polyurethane is used as the polymer is 4,4′-diisocyanatodicyclohexylmethane.

Further details and advantages of the invention result from the model reactions described below and a working example.

All chemicals used were used as commercially available and without further purification in the experiments conducted.

Nylon 6/6 pellets were purchased from Sigma Aldrich Corporation. The molecular weight of the compound was determined by gel permeation chromatography and quantified as 1.66 10⁵ g/mol. N-methylacetamide and 1,3,5-trioxane were each purchased at a purity of 99% from Sigma Aldrich Corporation, bismuth (III) trifluoromethanesulfonate and paraformaldehyde were purchased at a degree of purity for synthesis from Sigma Aldrich Corporation. Water was used fully deionized.

### First Model Reaction

In a first model experiment, the chemical reaction taking place in the method according to the invention was tested on a non-polymeric model molecule which comprises a structural unit having a nitrogen-carbonyl carbon bond. N-methylacetamide was selected as the model molecule.

For the experiment, one equivalent of N-methylacetamide was reacted with one equivalent of paraformaldehyde in the presence of bismuth triflate as catalytically active Lewis acid. The chemical reaction was carried out at a reaction temperature of 60° C. without solvent or with chloroform as solvent.

Without being bound to the theory, the inventors suspect that, in the chemical reaction carried out, the carbonyl carbon atom of the paraformaldehyde reacted with the nitrogen atom of the secondary amine of the N-methylacetamide in a nucleophilic addition reaction, almost completely converting the reaction starting materials to a hemiaminal. In a first reaction step, the free electron pair of the nitrogen atom of the secondary amine of N-methylacetamide nucleophilically attacked the positively polarized carbonyl carbon atom of paraformaldehyde here. The ensuing transition state comprised a molecule having positively charged nitrogen and an alkoxide anion. In a second reaction step, tautomerization of a hydrogen atom took place. The alkoxide anion deprotonated the formally quaternary nitrogen here, forming a hydroxyl group and a tertiary nitrogen. This is how the hemiaminal was finally formed.

It was found that without a catalyst in the conversion reaction of N-methylacetamide and paraformaldehyde to the hemiaminal, the reverse reaction to the reaction starting materials preferentially takes place at higher reaction temperatures before cleavage of the amide bond can occur.

In a subsequent reaction, the hemiaminal was further converted in the presence of bismuth triflate as catalytically active Lewis acid at a reaction temperature of 130° C. Acetic acid and formaldehyde-aldimine were formed as a result of concerted rearrangement here.

The yield of acetic acid was determined from a nuclear magnetic resonance spectroscopic signal relative to the reaction starting material N-methylacetamide. The yield was about 25%.

Two blank reactions were carried out to check the model reaction:

### Comparative Reaction A

Firstly, the reaction was carried out without formaldehyde or paraformaldehyde. That is, N-methylacetamide was mixed with 5 mol % bismuth triflate at a reaction temperature of 130° C. overnight. The resulting reaction product was a clear oil. If the amide had reacted, a cloudy mixture would have been expected. Accordingly, an examination of the reaction product by nuclear magnetic resonance spectroscopy without prior work-up revealed that only N-methylacetamide, but no hemiaminal and no acetic acid, were to be found in the reaction product.

### Comparative Reaction B

For a second blank reaction, the reaction was carried out without a catalyst, i.e. without Lewis acid. For this, N-methylacetamide was mixed with paraformaldehyde at a reaction temperature of 130° C. overnight. As is known from the literature, about 50% of the N-methylacetamide has been converted here to the hemiaminal (cf. Chem Ber. 1961, 1879). A further reaction, in which the hemiaminal was converted to acetic acid, did not take place without the presence of the Lewis acid acting as catalyst.

### Second Model Reaction

In a second model experiment, the chemical reaction of the first model reaction was investigated in more detail.

For this, one equivalent of N-methylacetamide was reacted with one equivalent of paraformaldehyde in the presence of 5 mol % Lewis acid on a 3 mmol scale. The reaction was carried out in a 10 ml pressure vessel without solvent at 130° C. reaction temperature for 23.5 hours. As Lewis acid, zinc triflate, aluminum triflate, cerium(III) triflate, scandium triflate, neodymium triflate, samarium triflate, yttrium triflate, lanthanum triflate, bismuth triflate or gadolinium triflate was used. It was found that the chemical reaction forms acetic acid and methylamine. According to a nuclear magnetic resonance spectroscopic investigation in deuterated water, the yield of acetic acid when bismuth triflate was used as the catalytic Lewis acid was 19%.

In a further experiment, one equivalent of N-methylacetamide was again reacted with one equivalent of paraformaldehyde on a 3 mmol scale in a 10 ml pressure vessel in the presence of 5 mol % bismuth triflate as the catalytically acting Lewis acid. The reaction was also carried out without solvent at a reaction temperature of 130° C., this time for 19 hours. Either one, two or three equivalents of water were added here. The added water caused a shift in the reaction equilibrium in favor of the reaction products. Accordingly, a nuclear magnetic resonance spectroscopic investigation of the reaction product in dimethyl sulfoxide showed that the yield of acetic acid could be increased to 25% by the addition of three equivalents of water.

### Working Example

In a subsequent experiment, the reaction according to the invention was then applied to a polymer.

For this purpose, polyamide 6.6 (226 mg, 1 mmol of repeating units, of the aforementioned nylon 6/6 pellets), paraformaldehyde (60 mg, 2 mmol), 1,3,5-trioxane (600 mg), bismuth triflate (33 mg, 0.05 mmol) and water (54 μL, 3 mmol) were initially mixed in a 10 ml pressure vessel. The reaction mixture was then stirred at 165° C. in a temperature-controlled heating block for 5 hours. The reaction mixture was then cooled, stirred into water and filtered off. The solid filter residue was then washed with small portions of water. Finally, the filter residue was dried for 30 minutes at a temperature of 70° C. In this way, 192 mg of reaction product were obtained, which corresponds to a reaction yield of 85%.

To quantify the depolymerization of the polymer used, which was cleaved with paraformaldehyde as activating reagent and bismuth triflate as catalytically active Lewis acid, the molecular weight distribution of the reaction starting material polyamide 6.6 and the molecular weight distribution of the reaction product obtained were investigated by ¹³C-nuclear magnetic resonance spectroscopy. Based on the ratios of characteristic carbon signals in the ¹³C-nuclear magnetic resonance spectroscopy, the molecular weight distribution can be determined. For the ¹³C nuclear magnetic resonance spectroscopy, the samples were dissolved in non-deuterated trifluoroethanol. The measurement of the reaction starting material polyamide 6.6 resulted in signals in the high-field range at 25.4; 26.4; 28.9; 36.0; 40.1 ppm. In the case of the reaction product, in turn, signals at 24.4; 25.3; 33.7; 36.1; 43.9 ppm were produced in the high-field range. The carbon signals were identified and assigned to the corresponding methylene groups of the terminal monomers via measurements of specially synthesized oligomeric hexamethylenediamine adipic acid amides. The signal ratios of the intact polyamide 6.6 polymer chain to the newly formed terminal monomer groups were in the ratio 1:0.31. This allowed a conversion of the amide groups present in the polymer of 24% to be concluded, which means a reduction in the average polymer chain lengths by a factor of 25.

1. A method for producing monomers and/or oligomers from a polymer comprising a structural unit having a nitrogen-carbonyl carbon bond, wherein the nitrogen-carbonyl carbon bond is cleaved in a chemical reaction with formaldehyde or paraformaldehyde as activating reagent, and wherein the reaction is carried out using a Lewis acid acting as catalyst.

2. The method as claimed in claim 1, wherein formaldehyde or paraformaldehyde is used stoichiometrically or substoichiometrically, wherein stoichiometric signifies that the amount of formaldehyde used or the repeating units of paraformaldehyde used is identical to the amount of repeating units of the polymer used or is a multiple or a whole multiple of the latter, and wherein substoichiometric signifies that the amount of formaldehyde used or the repeating units of paraformaldehyde used is only a fraction of the amount of repeating units of the polymer used.

3. The method as claimed in claim 1, wherein a metal catalyst is used as the Lewis acid.

4. The method as claimed in claim 3, wherein the metal catalyst comprises a metal from the group of: aluminum, boron, bismuth, cerium, iron, copper, lanthanum, magnesium, tin, titanium, zirconium or zinc.

5. The method as claimed in claim 3, wherein a metal triflate compound is used as metal catalyst.

6. The method as claimed in claim 1, wherein the chemical reaction is carried out in an organic solvent.

7. The method as claimed in claim 1, wherein water is used as an additive.

8. The method as claimed in claim 1, wherein the monomers and/or oligomers are produced in a reaction comprising a first and a second reaction stage.

9. The method as claimed in claim 8, wherein the first reaction stage is carried out at a reaction temperature of ≤100° C.

10. The method as claimed in claim 8, wherein the second reaction stage is carried out at a reaction temperature of ≤200° C.

11. The method as claimed in claim 8, wherein the first and the second reaction stage are carried out at a reaction temperature of ≤200° C.

12. The method as claimed in claim 8, wherein a hemiaminal is formed in the first reaction stage.

13. The method as claimed in claim 8, wherein a carboxylic acid and an aldimine are formed in the second reaction stage.

14. The method as claimed in claim 13, wherein the aldimine is further reacted to give an amine.

15. The method as claimed in claim 14, wherein the amine comprises an amine from the group of: pentamethylenediamine (PDA), hexamethylenediamine (HDA), isophoronediamine (IPDA), 2,4′- and 4,4′-diaminodicyclohexylmethane, 1,3- and 1,4-diaminocyclohexane, 1,3- and 1,4-bis (aminomethyl)cyclohexane (H6-XDA), 1,3- and 1,4-bis(aminomethyl)benzene (XDA) or bis(aminomethyl)norbornane (NBDA).

16. A monomer obtainable or produced by a method as claimed in claim 1.

17. The method as claimed in claim 5, wherein the metal triflate compound is bismuth triflate.

18. The method as claimed in claim 6, wherein the organic solvent is a polar organic solvent.

19. The monomer as claimed in claim 16, wherein the monomer comprises a carboxylic acid monomer and an aldimine monomer or an amine monomer.
